(19) United States
(12) Patent Application Publication
(10) Pub. No.: US 2023/0384323 A1
(43) Pub. Date: Nov. 30, 2023

(54) Method for Correction for Sample Volume

(71) Applicant: LMX MedTech LLC, Bloomfield, MI (US)

(72) Inventors: Michael J. Pugia, Pagosa Springs, CO (US); Jason M. Kulick, South Bend, IN (US)

(21) Appl. No.: 18/249,091

(22) PCT Filed: Oct. 15, 2021
(86) PCT No.: PCT/US21/55236

§ 371 Date: Apr. 14, 2023

Related U.S. Application Data
(60) Provisional application No. 63/092,811, filed on Oct. 16, 2020.

(51) Int. Cl. G01N 33/68

(52) U.S. Cl. CPC G01N 33/6893; G01N 2333/765

(57) ABSTRACT

A method for the analysis of one or more non-invasive sample biomarkers. Biomarkers within a normal concentration range may indicate not only the lack of disease, but also general health or lack of disease. These non-invasive sample biomarkers are corrected based on excretion of another non-invasive sample biomarker reflecting changes to the sample volume in a normal concentration range lacking disease. Method allows for indicating the existence of diseases, such as renal insufficiency, infections or other disease biomarkers by analyzing the concentration of biomarkers.

### CROSS REFERENCE TO RELATED APPLICATIONS

This application is the United States national phase of International Application No. PCT/US21/55236 filed Oct. 15, 2021, and claims priority to U.S. Provisional Patent Application No. 63/092,811, filed Oct. 16, 2020, the disclosures of which are hereby incorporated by reference in their entireties.

### BACKGROUND OF THE INVENTION

### Field of the Invention

The present disclosure relates to an accurate method for the analysis of one or more biomarkers in non-invasive sample fluids. Non-invasive sample fluids, like urine, saliva tears, sweat, nasal lavage, interstitial fluid, and other samples, are used estimate the biomarker concentrations in the blood without having to directly sample blood. Biomarkers outside of a normal concentration range indicate the presence of disease while those inside the normal concentration range indicate the lack of disease or general health. Non-invasive samples are prone to falsely predicting the biomarkers inside or outside of a normal concentration ranges due to variation in the amount of non-invasive sample volume secreted.

### Values Outside of Normal Range for

Biomarkers are consider indicators of disease (See list of target biomarkers in Pugia 63/006,833). Values inside of normal range are consider indicators of health or a lack of disease. Small change of values inside of normal range but moving towards abnormal are considered indicators of decreasing health and increasing disease. This is later rule can be followed for most biomarkers, including for inflammation markers like C-reactive protein (CRP) and Bikunin, nutrients and metals like ferritin, and iron, vitamins like vitamin B1, B6, B12 and D, enzymes like alkaline phosphatase and alanine transferase, proteins like albumin, globulins, hemoglobin, and all others, biomolecules like bilirubin, homocysteine, creatinine, cholesterols, and triglycerides, red blood cells (RBC), white blood cells (WBC) like lymphocytes, monocytes, neutrophils, eosinophils & basophils, platelets, metabolic markers like HbA1c, and glucose, acute phase markers like cytokines, chemokines and adhesion molecules, reactive oxidative species (ROS) and oxidative stress products, hormones, and many other biomarkers.

All biomarkers have a normal concentration range that is expected in the blood of humans and animals. Falsely predicting the blood concentrations can lead to a false determination health as in the case of a false negative or a false determination disease as in the case of false positive. Small changes within the normal range are often used as markers of healthy aging or self-care prior to disease and small changes are even harder to detect accurately. Values are even more difficult to accurately measure in non-invasive sample fluids which vary due to many conditions which impact the clearance of biomarkers into non-invasive sample fluids. These conditions include tissue damage, dehydration, excessive hydration, exercise, hyperthermia, blood pressure, infections, or others which can elevate or lower biomarkers in non-invasive sample fluids from concentrations expected in blood. Therefore, improvements are needed for correction biomarkers from non-invasive sample fluids as an indication of ideal health or progression toward disease.

The measurements of urinary biomarkers are an example of a non-invasive fluid impacted by the hydration status of the subject. Dehydration produce a concentrated urine sample with a high specific gravity of 1.030 and excessive hydration produces a dilute urine (diuresis) a water like specific gravity of 1.000, as discussed in, for example, Pugia, M. J., et al., Screening for proteinuria in Japanese schoolchildren: a new approach. Clin Chem Lab Med, 2000. 38(10): p. 975-82 (hereinafter “Reference 1”) Pugia, M. J., et al., Albuminuria and proteinuria in hospitalized patients as measured by quantitative and dipstick methods. J Clin Lab Anal, 2001. 15(5): p. 295-300 (hereinafter “Reference 2”).

The impact of metabolism on biomarkers is dependent on post-translation changes that occurs in vitro such as fragmentation and conjugation. However, once the primary forms of the urine biomarkers are identified, an assay can be constructed to measure biomarkers, for example, Pugia, M. J., et al., High-sensitivity dye binding assay for albumin in urine. J Clin Lab Anal, 1999. 13(4): p. 180-7 (hereinafter “Reference 3”). However, the concentrations of all biomarkers are greatly impacted by variations in sample volume and corrections are needed to reflect the actual rates of excretion of biomarkers from blood, as discussed in, for example, References 1-3.

For example, the volume of urine isn't constant but extremely variable with from a water like sample with total solids content to a highly concentrated sample with 35 fold greater total solids content. This 35-fold range of concentration impacts on measurements of biomarkers from blood, as discussed in, for example, References 1-3. The concentration of the biomarkers changes with volume of sample voided in the specimen collected. Urinary concentrations for biomarkers are best measured as rates of excretion over 24 h. The 24 h rates of excretion, e.g. the quantity for urinary constituents found per 24 h of urine collection, remain relatively constant. The 24 h rates of excretion for urine biomarkers reflect the blood levels and are therefore more important for diagnosis.

Similarly of saliva, tears and sweat are also extremely variable due to a variety hydration conditions from a water like sample with total solids content of 1.004 to a highly concentrated sample with a total solids content of 1.028 (Paper 1).

In practice, rates of excretion for biomarkers in non-invasive samples are difficult to measure as all samples must be gathered over long periods of time, e.g. 24 hours. The clinic measures the volume of the sample collected, and the concentration is expressed per volume of sample collected. For example, after the corrections of volume, sample volume is expected to be in a typical range, for example 1.5 L for urine per 24 hours, taken to represent a typical sample concentration for an adult, which is an of 1.010 to 1.015 specific gravity. Therefore, the measurement of rates of biomarkers excretion is quite labor intensive as all the samples must be collected combined and is rarely done in practice.

Collecting first morning samples is another means to reduce the impact of the volume of sample collected to better predict biomarker rates of excretion. For example, the first morning urine voided is collected for analysis after instructing a patient not to drink water before bed and until the morning and to collect the first void in the morning at least 4 to 8 hours from the last void. Here, the first-morning urine should typically be sufficiently concentrated (SG>1.020) to measure any biomarkers without risk of false negative due to a water like urine (SG<1.005) as discussed in, for example, Reference 1, Newman, D. J., et al., Urinary protein and albumin excretion corrected by creatinine and specific gravity. Clin Chim Acta, 2000. 294(1-2): p. 139-55 (hereinafter “Reference 4”), Pugia, M. J., et al., Screening school children for albuminuria, proteinuria and occult blood with dipsticks. Clin Chem Lab Med, 1999. 37(2): p. 149-57 (hereinafter “Reference 5”), Pugia, M. J., et al., Comparison of instrument-read dipsticks for albumin and creatinine in urine with visual results and quantitative methods. J Clin Lab Anal, 1998. 12(5): p. 280-4 (hereinafter “Reference 6”), Pugia, M., et al., Detection of low-molecular-weight proteins in urine by dipsticks. Clin Chim Acta, 2002. 326(1-2): p. 177-83 (hereinafter “Reference 7”), Shihabi, Z. K., R. P. Schwartz, and M. J. Pugia, Decreasing the variability observed in urine analysis. Ann Clin Lab Sci, 2001. 31(1): p. 99-102 (hereinafter Refernece 8”), and Wallace, J. F., et al., Multisite evaluation of a new dipstick for albumin, protein, and creatinine. J Clin Lab Anal, 2001. 15(5): p. 231-5 (hereinafter “Reference 9”). In this practice, there is no need to adjust for void volume or time of void for defining a first morning urine collected. However, theses first morning sample have pronounced diurnal effects and the first morning sample can over-estimate the amounts of biomarker as the sample. The total proteins (>15 mg protein/dL) and the solids content (SG>1.020) of these samples are higher values than expected which cause false positive results in the absence of disease (reference 4 and 5). Normalization with total protein or specific gravity does not improve biomarker correlation to the expected 24 h values as these values are not elevated to the same degree.

The first morning sample for saliva also have pronounced diurnal effects where the total protein and solids content of these samples are higher than expected. Normalization with total protein or specific gravity for saliva output does not improve the poor correlate of saliva CRP with blood values (Paper 2). Accounting for the time of day and sample volume improve biomarker correlation to the expected 24 h values (Paper 2). Without correction for excretion rate the correlation most markers, like CRP and Homocysteine, in saliva are poorly correlated to blood values (Paper 3). However timed collection with measure volumes are even more difficult to do in practice for sweat, tears and saliva.

Secondary biomarkers are another method often used to estimate the sample concentration so the sample volume can be accounted for. Urinary creatinine concentration is one commonly used biomarker for urinary concentration. Urinary creatinine concentration of 100 mg/dL being the typical for an average urine volume of 1.5 L over 24 h, as discussed, for example, in References 4-8. A highly dilute urine has SG of 1.005 and urinary creatinine concentration of mg/dL. The values are 100% lower than 24 h value. A random urine typically has a creatinine concentration between 50 mg/dL to 150 mg/dL values, as discussed in, for example, Reference 1, 4-9. Most first morning urines samples typically have creatinine value in this range. Highly dilute urine (a water like sample) can be reported as inadequate sample with a risk of false negative when the creatinine is less than 25 mg/dL, as discussed in, for example, References 2, 9, and Pugia, M. J., et al., Assay of creatinine using the peroxidase activity of copper-creatinine complexes. Clin Biochem, 2000. 33(1): p. 63-70 (hereinafter “Reference 10”). A highly concentrated urine is indicated by a creatinine value of 200 mg/dL (200% higher concentration than expected in a 24 h value) and 300 mg/dl (300% higher concentration than expected in a 24 h value).

In practice, the method of correction by creatinine ratio is prone to issues. Urine creatinine is very inaccurate at measuring diuresis or a dilute sample (reference 10). Highly dilute sample (SG<1.007) can have moderately elevated creatinine, and values require a second sample (Reference 9). There is a general lack of accuracy of the creatinine biomarker to predict smaller changes in urinary concentration (SG changes of 0.005). Creatinine biomarker is also highly predictive of the dilute urines, as discussed in, for example, References 6, 10, and Pugia, M. J., et al., Comparison of urine dipsticks with quantitative methods for microalbuminuria. Eur J Clin Chem Clin Biochem, 1997. 35(9): p. 693-700 (hereinafter “Reference 11”). Measurements of creatinine concentrations by immunoassay or optical chemistries do not correlate urinary concentration by specific gravity measurements in dilute urine. As a result, the uncorrected biomarker concentration is reported in favor of the biomarker ratio to creatinine concentration.

In practice, biomarkers that are expected to indicate disease or tissue damage, such as albumin cannot be used for corrections for sample volume, as diseases such as renal disease contribute to the albumin in urine, and normalized sample with albumin due to kidney disease will over prediction a highly dilute samples causing a false result. These biomarker ratios correct for sample concentration and reduce the impact of sample volume when compared to un-ratioed results, as discussed in, for example, References 1 and 4-9. The values of second biomarker, must not be indicative of disease of a patient, and are primarily only a measure of urinary concentration.

A solution for a more accurate way to correct the non-invasive sample concentration of biomarkers is needed. The solution needs to better to reflect the actual rates of excretion.

### SUMMARY OF THE INVENTION

In some non-limiting embodiment of the present disclosure uses immunoassays able to determine concentration levels of one or more biomarkers in non-invasive samples for indicating indicate the presence of disease while those value inside the normal concentration range indicate the lack of disease or general health, as well as one or more additional biomarkers in non-invasive samples for indicating changes to the sample volume when compared with concentration levels. The first biomarker concentrations may be corrected based on concentrations of a second biomarker to reflect the actual rates of excretion of the first biomarker more accurately. Concentration levels of the second biomarker may be indicative of disease, and indicating lack of sample validity for for ratioing. The concentrations levels of the all biomarker may outside the measurable range indicating lack of sample validity for for ratioing if below the measurable level.

In a non-limiting embodiment of the present disclosure, flags are used when the second biomarker is outside the concentration range reflecting the normal range. High concentration levels of the second biomarkers are indicative of a lack of sample validity for ratioing as disease is clearly indicate. In non-limiting embodiments, concentrations outside of a normal range, such as low or high concentration levels of the second biomarker, may be indicative of disease, tissue damage or other diseases. In other non-limiting embodiments, concentration levels of the first or second biomarker outside of the measurable range are reported for lack of sample validity for ratioing. In a non-limiting embodiment of the present disclosure, the second biomarker is a blood protein and able to measure vascular permeation of fluid into non-invasive samples. Values inside the normal concentration range of second biomarker may be used to reflect the volume of the sample.

In other non-limiting embodiments, second biomarker is with a molecular weight>50 to 70 kDa such as albumin.

In other non-limiting embodiments, concentration levels of the first biomarker, or the ratio the first biomarker to second biomarker outside of the normal range are indicative of disease. In other non-limiting embodiments, concentration levels of the first biomarker or the ratio the first biomarker to second biomarker inside of the normal range are indicative of health or lack of disease. In other non-limiting embodiments, concentration levels of the first biomarker or the ratio the first biomarker to second biomarker inside of the normal range but progressing toward being outside the normal range are indicative of loss health or progression toward disease.

Further preferred and non-limiting embodiments or examples are set forth in the following numbered clauses.

Clause 1: A method of determining concentration of at least one non-invasive sample biomarker comprising: introducing a sample into a well, wherein the sample comprises at least one biomarker; capturing the at least one biomarker; and determining that the at least one biomarker is indicative of lack of health.

Clause 2: The method of clause 1, further comprising determining concentration of the at least one biomarker indicating non-invasive sample volume and comparing the concentration of the at least one biomarker to a normal range.

Clause 3: The method of any of clauses 1-2, wherein a concentration of a first biomarker is corrected by a concentration of a second biomarker as to rates of excretion of the first biomarker.

Clause 4: The method of any of clauses 1-3, wherein concentrations of the first or second biomarkers outside of the normal concentration range are used to indicate a disease.

Clause 5: The method of any of clauses 1-4, wherein the sample is chosen from a group comprising human serum albumin, hydroxynonnel to human serum albumin, malondialedehyde to human serum albumin, uristatin, or bikunin.

Clause 6: The method of any of clauses 1-5, further comprising determining a concentration of the biomarker, wherein a concentration of albumin indicates the concentration of the biomarker.

Clause 7: The method of any of clauses 1-6, wherein concentrations of the first or second biomarkers inside of the normal concentration range indicate lack of disease or health.

Clause 8: The method of any of clauses 1-7, wherein concentrations indicate a degree of health or progression to disease.

Clause 9: The method of any of clauses 1-8, further comprising determining a concentration of the biomarker in non-invasive sample fluids, like urine, saliva, tears, sweat, nasal lavage, interstitial fluid, and other samples, are used to estimate the biomarker concentrations in the blood without having to directly sample blood.

These and other features and characteristics of the present disclosure, as well as the methods of operation and functions of the related elements of structures and the combination of parts will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 shows a schematic view of the analysis of biomarkers in non-invasive sample according to a non-limiting embodiment of the invention.

FIG. 2 shows a schematic view of a set of wells for capture and detection a biomarker according to a non-limiting embodiment of the invention.

FIG. 3 shows four immunoassays for urinary biomarkers according to a non-limiting embodiment of the invention.

### DESCRIPTION OF THE INVENTION

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

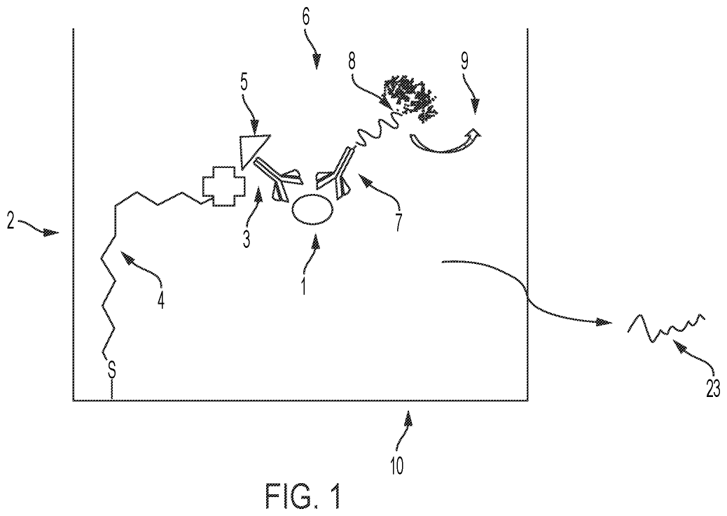

FIGS. 1 to 3 illustrate an embodiment of the present disclosure, where immunoassays are able to determine concentration levels of one or non-invasive sample biomarkers indicating the presence of disease while those inside the normal concentration range indicate the lack of disease or general health. Such can be indicated by levels of one or more additional non-invasive sample biomarkers that indicate changes in the non-invasive sample concentration when measured against concentration levels in the normal range of the additional non-invasive sample biomarkers.

FIG. 1 shows a schematic view of the present disclosure for analysis of biomarkers in non-invasive samples where the analyte (1) is captured in a well (2) by an affinity agent (3) either directly attached to the linkage arm (4) or bound to the linkage arm (4) via a high affinity label and capture agent (5). The analyte (1), such as a biomolecule or cell is captured by an affinity agent (3) after the addition of a sample (6) to the well. A second affinity agent (7) for a biomarker may be added and may attach to a reagent capable of generating a signal (8) after the removal of sample waste (23) from the well (2).

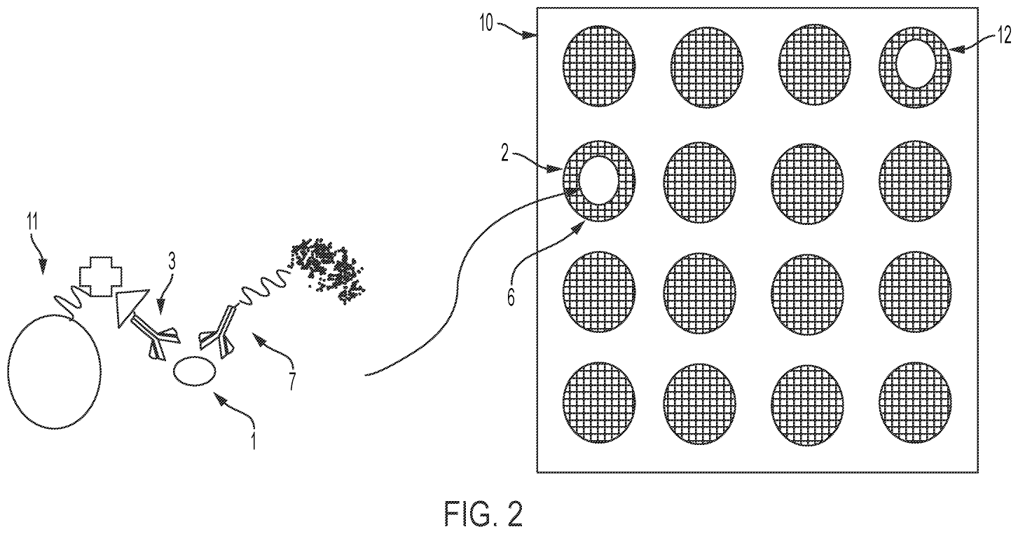

FIG. 2 shows a schematic view of a set of wells (10) able to capture and detect a biomarker, or an analyte (1), by addition of an affinity agent (3) for specific capture and detection (9). In a non-limiting example, a first biomarker may be a neutravidin microparticle (11) that is captured in the well (2). A first biomarker, such as a cell or biomolecule, is captured by affinity agent (3) and capture surface of the well (2) after the addition of reagents (3 and 7) and sample (6) to well (2) and followed by removal of waste (23) and by generation of signal (8) for determination of the first biomarker. One or more other wells (12) can be used for determination of a second biomarker via affinity agents and capture surface specific to the second biomarker.

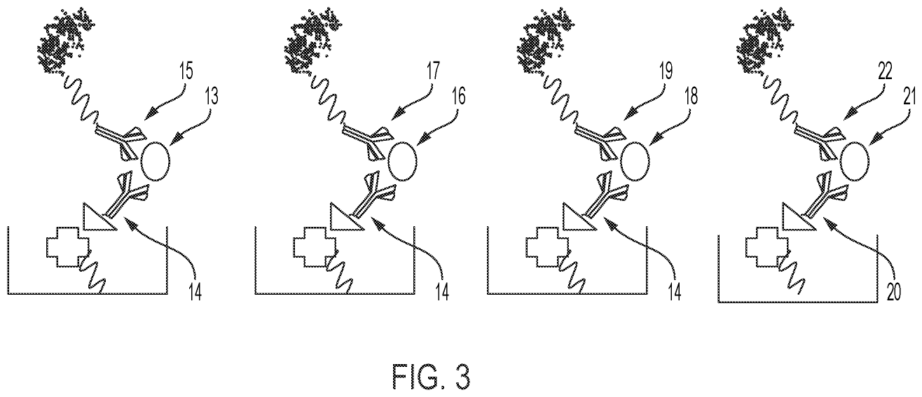

FIG. 3 shows four immunoassays for urinary biomarkers in accordance with a non-limiting embodiment of the invention as panel of biomarkers that indicate the presence of disease while those inside the normal concentration range indicate the lack of disease or general health. The biomarker (1) for measuring changes in the non-invasive sample concentration is shown by human serum albumin (HSA) (13) being bound by capture (14) and detection antibodies (15) against human serum albumin (HSA) (13). A first biomarker indicating the presence of disease or general health is the adduct of hydroxynonenal to human serum albumin (HNE-HSA) (16) being bound by capture agent (14) against human serum albumin (HSA) (13) and detection antibodies (17) against hydroxynonenal (HNE). A second biomarker indicating disease or general health is the adduct of malondialdehyde (MDA) to human serum albumin (MDA-HSA) (18) being bound by capture (14) against human serum albumin (HSA) and detection antibodies (19) against malondialdehyde (MDA). A third biomarker indicating health or lack of health is urinary trypsin inhibitor (Uristatin or Bikunin) (21) shown being bound by capture agent (20) and detection antibodies (22) against urinary trypsin inhibitor.

In some non-limiting embodiments of the present disclosure, the first step determines if the biomarker concentrations of the first and second biomarkers are within measurable range and able to produce valid ratio results. The next step determines if the second biomarker concentration is within the normal range and not at a level indicative of disease and able to produce valid a non-invasive sample concentration. Assuming results are reportable and not indicative of disease, the final step takes a ratio of the first biomarker with the second biomarker and reports the ratio results, which accurately reflects the actual rates of excretion of the first biomarker at levels indicative of lack of disease or general health. In some non-limiting embodiments of the present disclosure, there may be multiple first biomarkers, which indicate inflammation, oxidative stress, and other signs of general lack of lack of disease or general health.

In some non-limiting embodiments of the present disclosure, when the concentration levels of the first or second biomarkers outside the measurable ranges are measured, the limit of measurement ranges are reported. In some non-limiting embodiments of the present disclosure, if the second biomarker is at a level indicative of disease, then the ratio is not performed and the indication of disease is reported based on the second biomarker. In some non-limiting embodiments of the present disclosure, if the first biomarker is at a level indicative of disease, then the ratio is not performed and the indication of disease is reported based on the first biomarker. In some non-limiting embodiments of the present disclosure, the second biomarker may be an albumin, which may be indicative of disease, and the ratio is not performed.

In some non-limiting embodiment of the present disclosure, immunoassays are able to determine concentration levels of biomarkers by sandwich or competitive assay within dynamic ranges that allow measurement of concentrations determining disease or health. In practice, the invention can make use of optical immunoassay (OP-IA), electrochemical immunoassay (EC-IA) and mass spectrometric immunoassays (MS-IA) as an example previously described discussed in Pugia et al 63/006,833, 63/089,286, and 63/089,308, which are incorporated herein reference in their entireties.

### Example 1: Accurate Method to Correct the Urinary Concentration of Biomarkers to Reflect the Actual Rates of Excretion

To demonstrate the present disclosure, quantitative optical immunoassays (OP-IA) were developed for adduct of 4-Hydroxynonenal (HNE-HSA) to human serum albumin (HSA), adduct of Malondialdehyde (MDA) to HSA, and urinary trypsin inhibitor (also known as Uristatin or Bikunin) for lack of disease or general health. These biomarkers when above the normal range indicate a lack of health as important indicators of oxidative stress, as discussed in, for example, Akbari, M., et al., The Effects of Vitamin D Supplementation on Biomarkers of Inflammation and Oxidative Stress Among Women with Polycystic Ovary Syndrome: A Systematic Review and Meta-Analysis of Randomized Controlled Trials. Horm Metab Res, 2018. 50(4): p. 271-279 (hereinafter “Reference 12”), Mansournia, M. A., et al., The Effects of Vitamin D Supplementation on Biomarkers of Inflammation and Oxidative Stress in Diabetic Patients: A Systematic Review and Meta-Analysis of Randomized Controlled Trials. Horm Metab Res, 2018. 50(6): p. 429-440 (hereinafter “Reference 13”), Nasri, K., et al., The effects of vitamin D and evening primrose oil co-supplementation on lipid profiles and biomarkers of oxidative stress in vitamin D-deficient women with polycystic ovary syndrome: A randomized, double-blind, placebo-controlled trial. Endocr Res, 2018. 43(1): p. 1-10 (hereinafter “Reference 14”), Razavi, M., et al., The Effects of Vitamin D-K-Calcium Co-Supplementation on Endocrine, Inflammation, and Oxidative Stress Biomarkers in Vitamin D-Deficient Women with Polycystic Ovary Syndrome: A Randomized, Double-Blind, Placebo-Controlled Trial. Horm Metab Res, 2016. 48(7): p. 446-51 (hereinafter “Reference 15”), and Zheng, H. J., et al., The effect of probiotic and synbiotic supplementation on biomarkers of inflammation and oxidative stress in diabetic patients: A systematic review and meta-analysis of randomized controlled trials. Pharmacol Res, 2019. 142: p. 303-313 (hereinafter “Reference 16”) or inflammatory disease, as discussed in, for example, Pugia, M. J., et al., Immunological evaluation of urinary trypsin inhibitors in blood and urine: role of N- & O-linked glycoproteins. Glycoconj J, 2007. 24(1): p. 5-15 (hereinafter “Reference 17”), and Sasaki, M., et al., Measurement of the albumin content of urinary protein using dipsticks. J Clin Lab Anal, 1999. 13(5): p. 246-50 (hereinafter “Reference 18”) are elevated outside of their normal ranges to a level indicative of disease, as shown in Table 1. A second biomarker, namely human serum albumin (HSA), is used to illustrate a non-limiting embodiment of the present disclosure where this additional second biomarker indicates changes in the urine concentration when measured against the normal range. Concentration levels of first or second biomarkers outside a measurable range are not measurable other than to be reported at their limits of range by <= or >=.

Detection and capture affinity agents were made from HNE-HSA, MDA-HSA, uTi and HSA assays (See Materials below). Samples of urine were diluted 25-fold in PBS at pH 9 and a 100 μL sample added to a separate wells of polypropylene sample plate for each of four assays. Both detection and capture affinity agents were added at 0.5 to 2.0 μg/well to bind to HNE-HSA, MDA-HSA, uTi and HSA in samples using separate wells for each, and were incubated for 60 min at 37° C. The bound immunoassay complexes were transferred to a streptavidin coated high binding capacity microplates (Pierce) after blocked for 24 h with the blocking agent as described (Pugia Anal Chem 2021). The immunoassay complexes were bound to streptavidin coated microplates using separate wells for each sample by incubating for 5 min at 25° C. ELISA wells were then washed five with 200 μL TBS-T(0.05% Tween-20) in a EL406 plate washer (BioTek) and read on optical readers (BioTek) after addition of para-nitro phenyl phosphate as the signal. In this method, alkaline phosphatase is used to generate para-nitro phenol as the optical reporter from para-nitro-phenyl phosphate.

TABLE 1
Measurement ranges, normal ranges and levels
indicative of disease for biomarkers

| | | | Levels |
|---|---|---|---|
| | Measurable | Normal | indicative |
| Assay | Range | range | of disease |
| MDA-HSA | 0.5 to 8.0 mg/dL | 0.5 to 8.0 mg/dL | >4.0 mg/dL |
| HNE-HSA | 0.5 to 8.0 mg/dL | 0.5 to 8.0 mg/dL | >4.0 mg/dL |
| Uristatin | 0.5 to 12.0 mg/L | 0.5 to 7.5 mg/L | >7.5 mg/L |
| HSA | 0.5 to 80.0 mg/L | 0.5 to 40 mg/L | >40 mg/L |

### Materials:

| | |
|---|---|
| Detection | Polyclonal antibodies for recognizing Bikunin (LMX Med Tech MI), |
| affinity | malondialdehyde (MDA) (Abcam) and for recognizing 4-hydroxy-nonenal |
| agents | (HNE) (Abcam) were conjugated to ALP using the lightning link reagent |
| | (Abcam, MA). |
| Capture | Polyclonal antibodies recognizing Bikunin (LMX Med Tech MI), were |
| affinity | used for Bikuinin capture and those recognizing HSA were used for HSA, |
| agents | HNE-HSA and MDA-HSA capture (Bethyl Laboratories, PA) and each |
| | were conjugated to biotin-PEG4 using the EZ-Link NHS-conjugation kits |
| | (Thermo Fisher Scientific). The resultant antibody conjugates were stored |
| | at 4° C. |
| Antigens | Urinary Trypsin Inhibitor (SCPIC product code P205-1) and HSA (fraction |
| | V, Thermo Fisher) were used. Both malondialdehyde (MDA) and 4- |
| | hydroxy-nonenal (HNE) were reacted with human serum albumin to form |
| | the adduct of hydroxynonenal to human serum albumin (HNE-HSA) and |
| | the adduct of malondialdehyde to human serum albumin (MDA-HSA) |
| | followed by FPLC purification (AKTA prime, GE life sciences) |

Unless otherwise noted all other materials were purchased from Sigma Aldrich or Thermo Fisher Scientific.

All four methods were able to produce accurate and quantitative results in the measurable range and were also able to indicate when the biomarkers were outside the normal range, thus indicating disease such as shown in Table 1. Inside the normal range, lower biomarker values for MDA-HSA, HNE-HSA and Uristatin indicate a lack of disease or general health, with little inflammation or oxidative stress. Inside the normal range, lower HSA values indicate a lower urine concentration or low specific gravity (SG), and higher HSA values indicate a higher urine concentration or high specific SG value.

To demonstrate the correction for urine concentration according a non-limiting embodiment of the invention, the concentration of the MDA-HSA, HNE-HSA, HSA and uTi biomarkers were measured in low specific gravity (SG) (SG=1.005), medium SG (SG=1.010) and high SG (SG=1.030) urines. These changes in SG reflect changes in the excretion rate of biomolecules and urine volume, with high SG indicating 200% decrease in the excretion rate or 2× concentration due to reduced urine volume compared to medium SG urine concentration and low SG indicating 50% increase in excretion rate or 2× dilution due to increased urine volume compared to medium SG urine concentration.

Human serum albumin (HSA) values measured in the three urine pools of different SG were found to be 0.8 mg/L for low SG of 1.004, 1.7 mg/L for med SG of 1.011 and 5.2 mg/L for high SG of 1.019. Human serum albumin (HSA) was selected as an example the second biomarker for correction as it predicted urine concentration within its normal range of 0.5 to 40 mg/L. It is well known that HSA values less than 40 mg/L indicate health in the patient or a lack or albuminuria, while HSA values greater than 40 mg/L indicate disease, such as kidney disease or hypertension.

The uTi values in these pools were measured in three pools and found to be 5.0 mg/L for low SG, 4.8 mg/L for med SG and 10.2 mg/L for high SG. The uristatin levels of >7.5 mg/L indicate disease, e.g. infection. Therefore, uristatin (uTi) cannot be selected as, for example, the second biomarker for correction, as it did not predict urine concentration within its normal range of 0.5 to 7.5 mg/L. The MDA-HSA and HNE-HSA values were measured in three pools and also did not correlate to urine concentration (e.g. SG), and therefore could not be selected as an example of a second biomarker for urine correction. They were all within their normal range of 0.5 to 8.0 mg/L.

Using HSA ratioing of three biomarkers of panel assays of HNE-HSA, MDA-HSA and uTi could be corrected for urine concentration (e.g. SG) according to a non-limiting embodiment of the invention. This was performed by first confirming the biomarker concentrations of the first biomarkers (HNE-HSA, MDA-HSA and uTi) and the second biomarker (HSA) where all inside measurable ranges were able to produce a valid ratio result. If the second biomarker, HSA in this example, had concentrations outside the normal range indicating disease (>40 mg/L), then the disease level would be reported to indicate that sample was not from a healthy person and not able to produce valid ratio results. The final step is to ratio each of first biomarkers (HNE-HSA, MDA-HSA and uTi) to the second biomarker (HSA) and to report ratio results (mg biomarker 1/mg biomarker 2), which accurately reflect the actual rates of excretion of the first biomarker at levels indicative of disease or health.

To demonstrate the invention, eight levels of each analyte were added to three urine pools spanning the SG range to provide contrived samples that spanned the measurable ranges HNE-HSA, MDA-HSA and uTi assays (Table 1). The ratios of HNE-HSA to HSA (mg/mg), MDA-HSA to HSA(mg/mg) and uTi to HSA(mg/mg) were determined for each of eight levels. The sample bias between expected and reported concentrations was <18% for all ratio levels and within the acceptable limits for producing accurate and quantitative ratio results in contrived urine samples. This compared favorably to the sample bias between expected and reported concentration levels without ratioing of <21% across all samples.

For verification of the improvement, the panel assays were ratioed with 87 urine specimens collected from patients with diseases and healthy patients. All urine specimens were measured for specific gravity by TS meter to 0.001 SG and found to range from SG 1.004 to 1.030. Of these 87 samples, 21 had value of 40 mg/L HSA indicative of disease. These 21 samples had HSA values independent of urine SG as shown by a correlation coefficient of zero as they were samples of disease. Whereas the 66 samples below 40 mg/L HSA, had HSA values dependent on the urine as shown by a coefficient of R>0.8 to SG.

The impact of the albumin ratio according to the invention was measured by reducing the sample bias for MDA-HSA, HNE-HSA, and uTi across range of urine concentrations (SG 1.004 to 1.030). After performing the HSA ratio, the sample bias was improved to 19%, 11%, and 15% for HNE-HSA to HSA (mg/mg), MDA-HSA to HSA(mg/mg), and uTi to HSA(mg/mg) respectively. This is significantly improved from the bias observed without ratioing, which were 35%, 31% and 34% for HNE-HSA (mg/dL), MDA-HSA to HSA(mg/mg) and uTi to HSA(mg/mg) respectively. Additionally, without removing samples with 40 mg/L HSA indicative of disease, the ratio could not improve the sample bias for these samples SG 1.004 to 1.030.

The correction for urine volume for using urine biomarkers indicates changes in the urine concentration when measured against the normal range. It was somewhat un-expected that biomarkers known to be impacted by disease, such as HSA, could reduce the impact of urine concentration on other urine biomarkers when used in the normal range. While not bound to a mechanism of such reaction, it is believed that normal albumin excretion (e.g. low levels) is generally constant and changes in the normal range primarily reflect change in urine concentration (e.g. SG).

### Example 2: Accurate Method to Correct the Saliva Concentration of Biomarkers to Reflect the Actual Rates of Excretion

To demonstrate the present disclosure as a correction for saliva volume measures the adduct of 4-Hydroxynonenal (HNE-HSA) to human serum albumin (HSA) and human serum albumin (HSA) were used.

Human serum albumin (HSA) was picked from over 500 measurable proteins have found in human saliva as the vast majority are impacted in patients with cancer (Paper 4). The impact of cancer excludes these proteins from use as biomarker for correction sample volume. Human serum albumin (HSA) and amylase were the most abundant proteins. Both are in the 50-kDa molecular weight range ideal for measuring of sample volume due to vascular permeation of blood into saliva. However, amylases' main function is to hydrolyze the glycosidic bonds in starch molecules, converting complex carbohydrates to simple sugars, and its secretion is greatly impacted by diet, prohibiting it uses.

Human serum albumin (HSA) was selected as an example the second biomarker for correction as it predicted saliva concentration as it normal range is 0.1 to 0.4 g/L in saliva (Paper 5). HSA ratioing of HNE-HSA, was done to demonstrate the expected expect impact on correction from of saliva samples with volume SG of 1.005, 1.015 and 1.030 according to a non-limiting embodiment of the invention. These sample had observed HSA values of 0.03 g/L. 0.1 g/L and g/L respectively.

To demonstrate the invention, four levels of HNE-HSA, were added to these three salvia pools spanning at SG of 1.005, 1.015 and 1.030. The 1.030 sample had 2 fold higher concentration levels of HNE-HSA then the 1.015 sample which had 2 fold higher concentration levels of HNE-HSA than the 1.005 samples. All samples spanned the measurable ranges HNE-HSA, and HSA. The ratios of HNE-HSA to HSA (mg/mg), were determined all twelve levels. The values varied by 211% without correction by HSA The values varied with correction by HSA ranged compared favorably eliminating sample bias between expected and reported concentration levels with ratioing at <11% across all samples.

The correction for saliva volume for using saliva biomarkers indicates changes in the saliva concentration can be correct by HSA. Again, it was somewhat un-expected that biomarkers known to be impacted by disease, such as HSA, could reduce the impact of saliva concentration on other saliva biomarkers when used in the normal range. While not bound to a mechanism of action, it is believed that normal albumin excretion (e.g. in the absence of tissue damage) is generally constant and changes primarily reflect change in saliva concentration (e.g. SG) due to vascular permeability, allowing a better estimation of blood values into non-invasive fluid.

The impact of the albumin ratio according to the invention was measured by reducing the sample bias for MDA-HSA, HNE-HSA, and uTi across range of urine concentrations (SG 1.004 to 1.030). After performing the HSA ratio, the sample bias was improved to 19%, 11%, and 15% for HNE-HSA to HSA (mg/mg), MDA-HSA to HSA(mg/mg), and uTi to HSA(mg/mg) respectively. This is significantly improved from the bias observed without ratioing, which were 35%, 31% and 34% for HNE-HSA (mg/dL), MDA-HSA to HSA(mg/mg) and uTi to HSA(mg/mg) respectively. Additionally, without removing samples with 40 mg/L HSA indicative of disease, the ratio could not improve the sample bias for these samples SG 1.004 to 1.030.

1. A method of determining concentration of at least one biomarker in a non-invasive sample comprising:
introducing a sample into a well, wherein the sample comprises at least one biomarker;
capturing the at least one biomarker; and
determining that the at least one biomarker is indicative of lack of health.

2. The method of claim 1, further comprising determining concentration of the at least one biomarker indicating non-invasive sample volume and comparing the concentration of the at least one biomarker to a normal range.

3. The method of claim 1, wherein a concentration of a first biomarker is corrected by a concentration of a second biomarker to as to rates of excretion of the first biomarker.

4. The method of claim 3, wherein concentrations of the first or second biomarkers outside of the normal concentration range are used to indicate a disease.

5. The method of claim 1, wherein the sample is chosen from a group comprising human serum albumin, hydroxynonnel to human serum albumin, malondialedehyde to human serum albumin, uristatin, or bikunin.

6. The method of claim 1, further comprising determining a concentration of the biomarker, wherein a concentration of albumin indicates the concentration of the biomarker.

7. The method of claim 3, wherein concentrations of the first or second biomarkers inside of the normal concentration range are used to indicate lack of disease or health.

8. The method of claim 3, wherein concentrations of the first or second biomarkers inside of the normal concentration range are used to indicate a degree of health or progression to disease.

9. The method of claim 1, further comprising determining a concentration of the biomarker in non-invasive sample fluids, like urine, saliva tears, sweat, nasal lavage, interstitial fluid, and other samples, are used to estimate the biomarker concentrations in the blood without having to directly sample blood.
